(19) United States
(12) Patent Application Publication
(10) Pub. No.: US 2007/0026508 A1
(43) Pub. Date: Feb. 1, 2007

(54) Enzymatic method of making 1,2-diol derivatives and their esters

(75) Inventors: Soon Ook Hwang, Daejeon (KR); Do Hoon Kim, Daejeon (KR); Hye Youn Ryu, Daejeon (KR)

Correspondence Address: IPLA P.A., 3580 WILSHIRE BLVD., 17TH FLOOR, LOS ANGELES, CA, 90010, US

(73) Assignee: ENZYTECH, LTD., DAEJEON (KR)

(21) Appl. No.: 10/554,962

(22) PCT Filed: Apr. 30, 2004
(86) PCT No.: PCT/KR04/01005

§ 371 Date: Oct. 28, 2005

(30) Foreign Application Priority Data
May 2, 2003 (KR) 10-2003-0028245

(51) Int. Cl. C12P 7/62; C12P 7/20

(52) U.S. Cl. 435/135; 435/159

(57) ABSTRACT

The present invention relates to a new process for the preparation of optically active alcohols represented by the general formula 2 and their esters represented by the general formula 3 by enzymatic method from racemic alcohols represented by the general formula 1 in scheme 1. In more details, this invention relates to a process for producing optically active alcohols and their esters which are used as pharmaceutical intermediates from alcohols represented by the general formula 1 by stereospecific transesterification of secondary hydroxyl group using lipases as catalyst with acyl donors in organic solvent or with acyl donors only without using organic solvent. According to this invention, optically active alcohols and esters of high optical purity in high yield can be produced by transforming the primary hydroxyl group of 1,2-diols by tosyl group and transesterifying the secondary hydroxyl group stereospecifically by lipase.

### TECHNICAL FIELD

The present invention relates to a new process for the preparation of optically active 1,2-diol derivatives and their esters by reacting the secondary hydroxyl group of racemic 1,2-diols represented by the general formula 1 stereospecifically using acyl donors in the organic phase or using acyl donors in non-solvent phase by enzymatic method.

This invention relates to a process for the products of high yield and optical purity by reacting 1,2-diol derivatives stereospecifically using acyl donors such as vinyl acetate, vinyl propionate and isopropenyl acetate, and lipases as catalysts.

Racemic alcohols represented by the general formula 1 in scheme 1 are composed of (S)-alcohols and (R)-alcohols respectively and they are used as intermediates in preparing important pharmaceuticals.

There are some enzymatic methods to prepare optically active 1,2-diols. In most cases, the primary hydroxyl group is transformed by other functional group and secondary hydroxyl group is hydrolyzed or esterified stereospecifically.

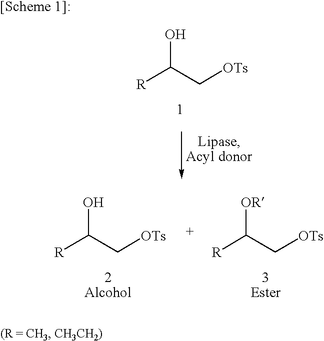

### BACKGROUND ART

Hamaguchi et al. obtained (S)-2-hydroxy-3-chloropropyl p-toluenesulfonate (99% ee) and (R)-2-acetoxy-3-chloropropyl p-toluenesulfonate (99%ee) by the hydrolysis of racemic 2-acetoxy-3-chloropropyl p-toluenesulfonate using LPL from Amano Inc. as biocatalyst (see Agric. Biol. Chem., 50(2), 375-380 (1986)) and Chen et al. obtained (S)-ester (97% ee) and (R)-alcohol (70% ee) by transesterification of 1-chloro-3-tosyloxypropane-2-ol using P-30 lipase from Amano Inc. as biocatalyst (see J. Chem. Soc. Perkin Trans I, 2559-2561(1990)).

On the other hand, Kim and Choi obtained (R)-2-hydroxy-3-chloropropyl tritylate (Yield 54%, 72% ee) and (S)-2-acetoxy-3-chloropropyl tritylate (Yield 43%, 98% ee) by the transesterification of 2-hydroxy-3-chloropropyl tritylate using PS lipase and vinyl acetate as the acylating agent in toluene (see J. Org. Chem., 57: 1605-1607 (1992)).

### DISCLOSURE OF INVENTION

Instead of Kim and Choi's method transforming primary hydroxyl group of 1,2-diols such as 1,2-propanediol or 1,2-butanediol by trityl group in the present invention, the primary hydroxyl group of 1,2-diols was transformed by tosyl group and the enzymatic esterification was carried out.

According to this invention, alcohols and the corresponding esters of high optical purity can be obtained in high yield as shown in the conventional method, furthermore, alcohols and their esters can be transformed easily in the next step by using tosyl group

Accordingly, the objectives of this invention are not only to transform 1,2-diols into racemic alcohols represented by the general formula 1 and to produce alcohols and their esters of high optical purity in high yield but also to transform optically active alcohols of the general formula 2 and their esters of the general formula 3 easily in the next synthetic step, thus this invention is considered better than the other conventional methods.

For the above objectives, the present invention consists of the process for reacting alcohols represented by the general formula 1 stereospecifically by lipase using acylating agent in organic solvent or using acylating agent only without organic solvent.

This invention is explained in more detail as follows.

As mentioned above, in this invention, lipase is added to alcohols represented by the general formula 1 without any pretreatment and transesterification is carried out as shown in scheme 1.

For the lipase, commercially available ones and, if necessary, home-made ones can be used. Non-limiting examples of the commercially available lipase include Novozyme 435 from Novo Ltd. and those manufactured by Amano Inc. such as PS, PS-D, PS-C and AK lipase. After reaction, optically active alcohols and their esters are separated by known methods such as solvent extraction.

Optically active 2-hydroxypropyl p-toluenesulfonate were determined by a HPLC (Lab Alliance Inc. Model 201) equipped with chiral column (Chiralcel OB-H, Daicel) using hexane and isopropyl alcohol mixture (80:20) as mobile phase. The Absorbance was 220 nm and flow rate was 0.65 ml/min. The typical retention time of the components in this invention was as follows:

(S)-2-hydroxypropyl p-toluenesulfonate—19.6 min

(R)- 2- hydroxypropyl p-toluenesulfonate—24.9 min

(R)-2-acetoxypropyl p-toluenesulfonate—38.9 min

Analytical condition of optically active 2-hydroxybutyl p-toluenesulfonate was the same as that of 2-hydroxypropyl p-toluenesulfonate except that flow rate was 0.45 ml/min. The typical retention time of the components in this invention was as follows:

(S)-2-hydroxybutyl p-toluenesulfonate—24.9 min

(R)-2-hydroxybutyl p-toluenesulfonate—27.9 min

(R)-2-acetoxybutyl p-toluenesulfonate—49 min

And racemic 2-hydroxypropyl p-toluenesulfonate and 2-hydroxybutyl p-toluenesulfonate thus synthesized were confirmed by FT-NMR (Burker Inc., Model DPX300) respectively and the resuls are as follows:

2-hydroxypropyl p-toluenesulfonate:

¹H-nuclear magnetic resonance (¹H-NMR)(CDCl₃):

δ 7.72(d, 2H), 7.28(d, 2H), 3.78 to 3.97(m, 3H), 3.06(bs, 1H), 2.38(s, 3H), 1.09(d, 3H)

2-hydroxybutyl p-toluenesulfonate:

¹H-nuclear magnetic resonance (¹H-NMR)(CDCl₃):

δ 7.8(d, 2H), 7.39(d, 2H), 3.78 to 4.08(m, 3H), 2.47(s, 3H), 1.46 to 1.51(m, 2H), 0.92 to 0.97(t, 3H)

A better understanding of the present invention may be obtained through the following examples which are set forth to illustrate, but are not to be construed as the limit of the present invention.

### EXAMPLE 1

1,2-propanediol (7.6 ml) was dissolved in dichloromethane (50 ml), 4-dimethylaminopyridine (0.49 g) and p-toluenesulfonyl chloride (24.7 g) were added to it respectively and maintained at 0˜5° C. And triethylamine (13.16 ml) was added dropwise for 30 minutes to 1 hour under nitrogen atmosphere. Then the reaction was carried out for 24 hours at room temperature. After reaction, the reaction mixture was poured into an ice-water mixture and extracted with dichloromethane. The combined organic phase was dried over and 2-hydroxypropyl p-toluenesulfonate (17.3 g, Yield 73%) was obtained and confirmed by FT-NMR.

Vinyl acetate (0.1 ml) and t-butylmethylether (4.9 ml) were placed in a 15 ml vial. Then, racemic 2-hydroxypropyl p-toluenesulfonate (0.05 g) and PS lipase (0.2 g) were added to the mixture. The reaction was carried out for 66 hours at 30° C. and (S)-hydroxypropyl p-toluenesulfonate (72.0% ee) and the corresponding (R)-2-acetoxypropyl p-toluenesulfonate (99% ee) were obtained at 44.0% conversion.

### EXAMPLE 2

Instead of 1,2-propanediol, 1,2-butanediol (2.25 ml) was dissolved in dichloromethane (50 ml), and the synthesis was performed as shown in Example 1.

After synthesis, 2-hydroxybutyl p-toluenesulfonate (4.95 g, Yield 81%) was obtained and confirmed by FT-NMR.

2-Hydroxybutyl p-toluenesulfonate was used instead of 2-hydroxypropyl p-toluenesulfonate in Example 1 and after 96 hours of reaction, (S)-2-hydroxybutyl p-toluenesulfonate (99.0% ee) and (R)-2-acetoxybutyl p-toluenesulfonate (99.0% ee) were obtained at 47.1% conversion.

### EXAMPLES 3-4

Enzymatic transesterification of 2-hydroxypropyl p-toluenesulfonate was carried out using lipases as shown in Table 1 instead of PS lipase in Example 1. The results are shown in Table 1.

TABLE 1

| | | | | % ee for (S)-2- | % ee for (R)-2- |
|---|---|---|---|---|---|
| | | Reaction | Conversion | hydroxypropyl p- | actoxypropyl p- |
| Example | Lipase | Time(hr) | (%) | toluenesulfonate | toluenesulfonate |
| 3 | CAL | 0.5 | 54.0 | 99.7 | 99.0 |
| 4 | AK | 19 | 55.6 | 99.6 | 99.0 |

### EXAMPLES 5-6

Enzymatic transesterification of 2-hydroxypropyl p-toluenesulfonate was carried out using acylating agents as shown in Table 2 instead of vinyl acetate. The results are shown in Table 2.

TABLE 2

| | | | | % ee for (S)-2- | % ee for (R)-2- |
|---|---|---|---|---|---|
| | Acylating | Reaction | Conversion | hydroxypropyl p- | propyloxypropyl p- |
| Example | agent | time(hr) | (%) | toluenesulfonate | toluenesulfonate |
| 5 | vinyl | 20 | 60.0 | 99.5 | 99.0 |
| | propionate | | | | |
| | | | | % ee for (R)-2- | % ee for for (S)- |
| | Acylating | Reaction | Conversion | hydroxypropyl p- | 2-acetoxypropyl p- |
| Example | agent | time(hr) | (%) | toluenesulfonate | toluenesulfonate |
| 6 | isopropenyl | 21 | 48.2 | 74.5 | 99.0 |
| | acetate | | | | |

### EXAMPLES 7-9

Enzymatic transesterification of 2-hydroxypropyl p-toluenesulfonate was carried out using the following solvents as shown in Table 3 instead of t-butylmethylether. The results are shown in Table 3.

TABLE 3

| | | | | % ee for (S)-2- | % ee for (R)-2- |
|---|---|---|---|---|---|
| | Organic | Reaction | Conversion | hydroxypropyl p- | acetoxypropyl p- |
| Example | solvent | time(hr) | (%) | toluenesulfonate | toluenesulfonate |
| 7 | diethylether | 66 | 44.5 | 73.0 | 99.0 |
| 8 | isopropylether | 20 | 54.1 | 99.7 | 99.0 |
| 9 | toluene | 15 | 53.1 | 97.1 | 99.0 |

### EXAMPLES 10-12

The acylating agents as shown in Table 4 were used in the reaction instead of organic solvent. The reaction was carried out as shown in Example 1 using AK lipase instead of PS lipase. The results are shown in Table 4.

TABLE 4

| | | | | % ee for (S)-2- | % ee for (R)-2- |
|---|---|---|---|---|---|
| | Acylating | Reaction | Conversion | hydroxypropyl p- | acetoxypropyl p- |
| Example | agent | time(hr) | (%) | toluenesulfonate | toluenesulfonate |
| 10 | vinyl acetate | 20 | 55.4 | 99.0 | 99.0 |
| 11 | isopropenyl | 21 | 50.6 | 84.6 | 99.0 |
| | acetate | | | | |
| | | | | % ee for (S)-2- | % ee for (R)-2- |
| | Acylating | Reaction | Conversion | hydroxypropyl p- | propyloxypropyl p- |
| Example | agent | time(hr) | (%) | toluenesulfonate | toluenesulfonate |
| 12 | vinyl | 20 | 53.8 | 99.0 | 99.0 |
| | propionate | | | | |

### INDUSTRIAL APPLICABILITY

In accordance with this invention, the starting material can be synthesized at lower cost by simple method. With using lipase, alcohols and their esters of high optical purity could be produced in high yield. Therefore it is a very useful process on the industrial scale.

What is claimed is:

1. A process for preparing optically active alcohols represented by the general formula 2 and their esters represented by the general formula 3 from racemic alcohols represented by the general formula 1, whose secondary hydroxyl group is esterified stereospecifically by lipase with using acylating agent in the organic phase or using acylating agent only in non-solvent phase.

2. The process according to claim 1, wherein R in the general formula 1 is an alkyl group containing 1 to 2 carbon atoms.
